(19) United States
(12) Patent Application Publication
(10) Pub. No.: US 2026/0306735 A1
(43) Pub. Date: Oct. 8, 2026

(54) DRUG INFUSION DEVICE FOR TUMOR INTERVENTIONAL THERAPY

(71) Applicant: GUANGZHOU INSTITUTE OF CANCER RESEARCH, THE AFFILIATED CANCER HOSPITAL, GUANGZHOU MEDICAL UNIVERSITY, Guangzhou (CN)

(72) Inventors: Guoshuo CHEN, Guangzhou (CN); Yingwen ZHU, Guangzhou (CN); Jiajun LI, Guangzhou (CN)

(21) Appl. No.: 19/215,284

(22) Filed: May 21, 2025

Related U.S. Application Data
(63) Continuation of application No. PCT/CN2025/087418, filed on Apr. 7, 2025.

(51) Int. Cl. A61M 5/14; A61M 5/158; A61M 5/168; A61M 25/02

(52) U.S. Cl. CPC A61M 5/1415; A61M 5/1413; A61M 5/158; A61M 5/16813; A61M 25/02; A61M 2005/1416; A61M 2005/1585; A61M 2005/1586; A61M 2025/0206; A61M 2025/024; A61M 2025/028; A61M 2205/3306; A61M 2230/30

(57) ABSTRACT

Disclosed is a drug infusion device for tumor interventional therapy, including a fixing base, a rotating support frame, a locking assembly, an infusion apparatus and a storage table. The fixing base is circumferentially fixed to an outer side of the human body; the rotating support frame is slidably connected to the fixing base; the locking assembly is used for locking the rotating support frame with the fixing base; the infusion apparatus is mounted on the rotating support frame for infusing drug; and the storage table is communicated with the infusion apparatus. By wearing the fixing base close to a patient's tumor site, the infusion apparatus is attached to a blood vessel to be punctured, realizing accurate drug infusion for tumor interventional therapy; and the locking assembly is matched with the rotating support frame, effectively preventing external force interference on the infusion apparatus, and improving infusion stability and safety.

### CROSS-REFERENCE TO RELATED APPLICATIONS

This application is a continuation of PCT/CN 2025/087418, filed on Apr. 7, 2025, the entire contents of which are incorporated herein by reference.

### TECHNICAL FIELD

The present disclosure relates to the technical field of drug infusion devices, and in particular to a drug infusion device for tumor interventional therapy.

### BACKGROUND

Tumor interventional therapy, a non-surgical method that directly treats tumors, typically includes various techniques under the guidance of imaging, such as percutaneous puncture, catheter insertion, or laparoscopy. This treatment method can deliver anticancer drugs, chemotherapeutic agents, or other therapeutic substances directly into tumor tissues, maximizing local therapeutic efficacy, and minimizing damage to surrounding healthy tissues.

In related art, conventional approaches such as direct catheter insertion often fail to reach or close to tumor sites. Particularly when the tumor sites are located near critical organs including heart, brain and the like, standard puncture approaches carry relatively high risks. During drug infusion, if a patient exhibits significant body movement and the infusion device lacks effective fixation mechanisms, particularly at body contact points where only medical adhesive tape is typically used for stabilization, it is difficult to provide effective protection against accidental external forces, thereby easily disrupting the infusion process and increasing the risk of injury.

### SUMMARY

The present disclosure provides a drug infusion device for tumor interventional therapy for solving the defects in the related art that the drug infusion device for tumor interventional therapy has low safety and inaccurate puncture position

On the one hand, the present disclosure provides a drug infusion device for tumor interventional therapy, including a fixing base, a rotating support frame, a locking assembly, an infusion apparatus and a storage table.

The fixing base is circumferentially fixed to an outer side of the human body; the rotating support frame is slidably connected to the fixing base; the locking assembly is used for locking the rotating support frame with the fixing base; the infusion apparatus is mounted on the rotating support frame for infusing drug; and the storage table is communicated with the infusion apparatus using a catheter for storing drug.

According to the drug infusion device for tumor interventional therapy provided by the present disclosure, the fixing base includes a flexible bottom plate, a sliding rod, a buckle strap and two flexible straps; the two flexible straps are fixedly connected to two ends of the flexible bottom plate, and the sliding rod is fixedly connected to the flexible bottom plate; and the buckle strap is fixedly connected to one of the flexible straps for locking the two flexible straps.

According to the drug infusion device for tumor interventional therapy provided by the present disclosure, the rotating support frame includes a top support frame, a rotating table and a clamping assembly, one end of the top support frame is slidably connected to the fixing base, and the other end is rotatably connected to the rotating table; and the clamping assembly is fixedly connected to the rotating table.

According to the drug infusion device for tumor interventional therapy provided by the present disclosure, the top support frame includes a four-link structure, a sliding sleeve I, and a sliding sleeve II; the sliding sleeve I is movably connected to the fixing base; the sliding sleeve II is rotatably connected to the fixing base; two ends of the four-link structure are rotatably connected to the sliding sleeve I and the sliding sleeve II; and a movable rod of the four-link structure is rotatably connected to the rotating table.

According to the drug infusion device for tumor interventional therapy provided by the present disclosure, the clamping assembly includes a sliding support frame, a drive arm and clamp plates, and the sliding support frame is fixedly connected to the rotating table; the clamp plates are slidably connected to the sliding support frame; and the drive arm is mounted on the sliding support frame for driving the clamp plates to clamp or release.

According to the drug infusion device for tumor interventional therapy provided by the present disclosure, the locking assembly includes a direction locking structure and position locking structures; the direction locking structure includes an outer spline and a locking block, the outer spline is slidably connected to the fixing base, and an inner spline groove bonded with the outer spline is disposed inside the sliding sleeve I; two sides of the locking block are slidably connect to the outer spline and the fixing base; the position locking structure includes a locking bolt and a limiting block; and the locking bolt passes through the sliding sleeve I and is screwed with the sliding sleeve I, and the limiting block is fixedly connected to the locking bolt.

According to the drug infusion device for tumor interventional therapy provided by the present disclosure, the infusion apparatus includes a puncture needle, a flow guide needle, a driver, a limiting stopper and a sealing structure; the puncture needle is used for puncturing a patient's blood vessel; the flow guide needle is arranged inside the puncture needle; the driver is used for driving the flow guide needle into or out of the blood vessel; the limiting stopper is used for limiting the flow guide needle, and causing at least one end of the flow guide needle to be located in the puncture needle; and the sealing structure is arranged inside a gap between the flow guide needle and the puncture needle.

According to the drug infusion device for tumor interventional therapy provided by the present disclosure, the flow guide needle includes a limiting end and an outlet end; a through hole is disposed at the limiting end; a valve is arranged at one end of the through hole; and a fiber optic sensor is mounted at the outlet end.

According to the drug infusion device for tumor interventional therapy provided by the present disclosure, the limiting stopper includes a spring and a jacking block; the spring is mounted inside the puncture needle, and an inner diameter of the spring is smaller than a maximum outer diameter of the limiting end; and the jacking block is mounted on an inner side of the spring for opening the valve.

According to the drug infusion device for tumor interventional therapy provided by the present disclosure, the puncture needle includes a needle head, a fixing block, a connecting pipe, a handheld portion and guide pipes; the needle head is fixedly connected to and communicated with the fixing block; the handheld portion is fixedly connected to the fixing block; the connecting pipe is communicated with the fixing block; the guide pipes are fixedly connected inside the connecting pipe; the driver includes a knob, a connecting rod, a sealing ring and two roller wheels, and the knob is mounted on an outer wall of the connecting pipe; and one end of the connecting rod is fixedly connected to the knob, the other end passes through the connecting pipe and is fixedly connected to one of the roller wheels, the other roller wheel is rotatably connected inside the connecting pipe, and the two roller wheels are arranged in parallel and symmetrically fitted on two sides of the flow guide needle.

In the present disclosure, by wearing the fixing base close to a patient's tumor site, the infusion apparatus is attached to a blood vessel position to be punctured through the rotating support frame, realizing accurate drug infusion for tumor interventional therapy; and the locking assembly is matched with the rotating support frame, which can effectively prevent external force interference on the infusion apparatus, improving infusion stability and safety.

In the present disclosure, during drug infusion, the driver moves the flow guide needle into the blood vessel, causing the outlet end of the flow guide needle to close to the tumor site. At this time, the limiting end of the flow guide needle is abutted against the spring, and the jacking block is driven to open the valve, causing the flow guide needle to be communicated with the puncture needle, thereby ensuring drug infusion through the flow guide needle to the tumor site. In the present disclosure, the accurate drug infusion for tumor interventional therapy is not only achieved, but also adjustable components including the flow guide needle and top support frame are arranged, causing the infusion arrangement to be applicable to the majority of the population, while effectively preventing drug waste during the infusion, thereby reducing therapeutic costs.

### BRIEF DESCRIPTION OF THE DRAWINGS

To more clearly illustrate the technical solutions in the present disclosure or in the related art, a brief description of the drawings required to be used in the description of the embodiment or the related art is presented below. Obviously, the drawings described below are some embodiments of the present disclosure. For those ordinary skilled in the art, other drawings may be obtained without creative efforts.

FIG. 1 is a schematic diagram showing a perspective structure of a drug infusion device for tumor interventional therapy according to an embodiment of the present disclosure;

FIG. 2 is a schematic diagram showing a partial perspective structure of the drug infusion device for tumor interventional therapy according to an embodiment of the present disclosure;

FIG. 3 is a schematic diagram showing a perspective structure of a rotating support frame in FIG. 1;

FIG. 4 is a schematic diagram showing a front view of a puncture needle in FIG. 1;

FIG. 5 is a schematic diagram showing a cross-sectional structure of the puncture needle in FIG. 4 along an A-A direction;

FIG. 6 is a schematic diagram showing a cross-sectional structure of a flow guide needle in FIG. 1;

FIG. 7 is a schematic diagram showing a cross-sectional structure of the flow guide needle in FIG. 1 located within the puncture needle;

FIG. 8 is an enlargement schematic diagram showing a structure at B in FIG. 7; and

FIG. 9 is an enlargement schematic diagram showing a structure at C in FIG. 7.

### REFERENCE NUMERALS AND DENOTATIONS THEREOF

1—fixing base; 11—flexible bottom plate; 12—sliding rod; 13—buckle strap; 14—flexible strap; 2—rotating support frame; 21—top support frame; 211—four-link structure; 212—sliding sleeve I; 213—sliding sleeve II; 22—rotating table; 23—clamping assembly; 231—sliding support frame; 232—drive arm; 233—clamp plate; 3—locking assembly; 31—direction locking structure; 311—outer spline; 312—locking block; 32—position locking structure; 321—locking bolt; 322—limiting block; 4—infusion apparatus; 41—puncture needle; 411—needle head; 412—fixing block; 413—connecting pipe; 414—handheld portion; 415—guide pipe; 42—flow guide needle; 421—limiting end; 422—outlet end; 423—valve; 424—fiber optic sensor; 43—driver; 44—limiting stopper; 441—spring; 442—jacking block; and 45—sealing structure.

### DETAILED DESCRIPTION

In order to make the purpose, technical solution and advantages of the present disclosure more clear, the technical solutions in the embodiment of the present disclosure are further described clearly and completely below in combination with the accompanying drawings. Obviously, the embodiments described are only some, rather than all embodiments of the present disclosure. Based on the embodiment of the present disclosure, all other embodiments obtained by those ordinary skilled in the art without creative efforts fall within the scope of protection of the present disclosure.

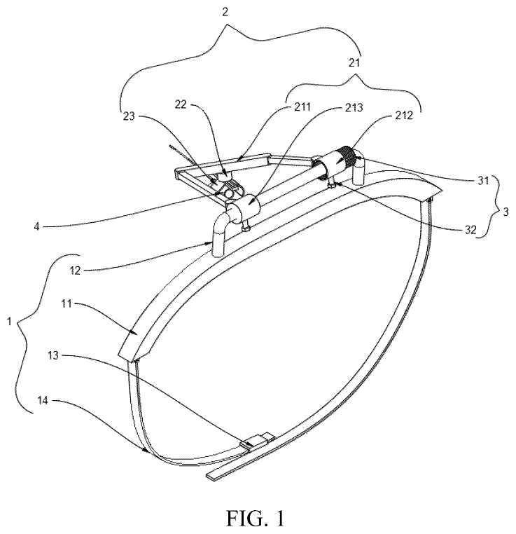

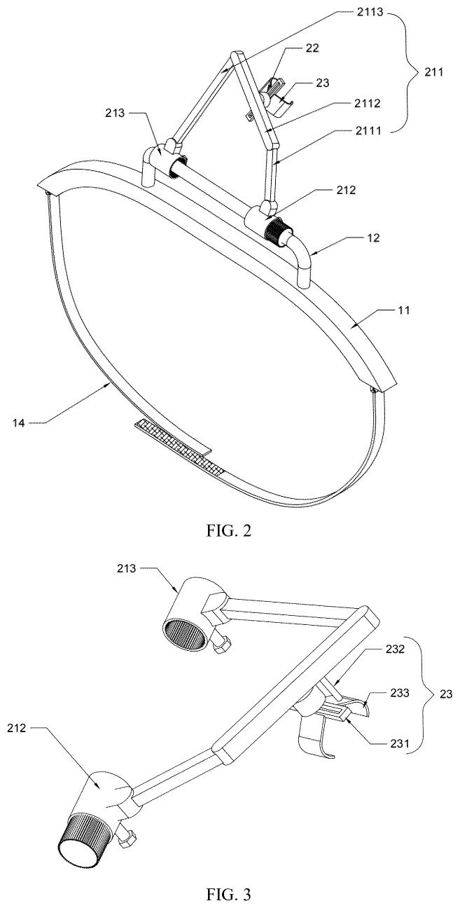

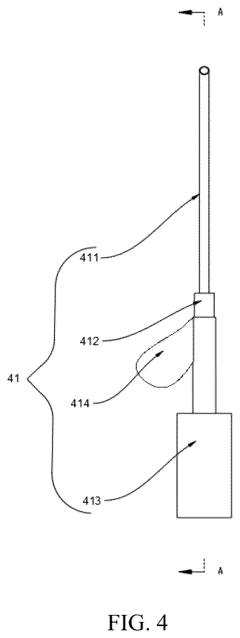

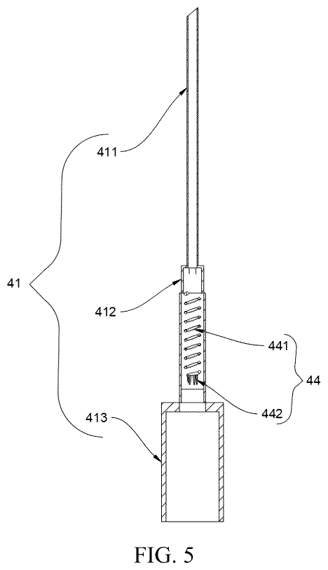

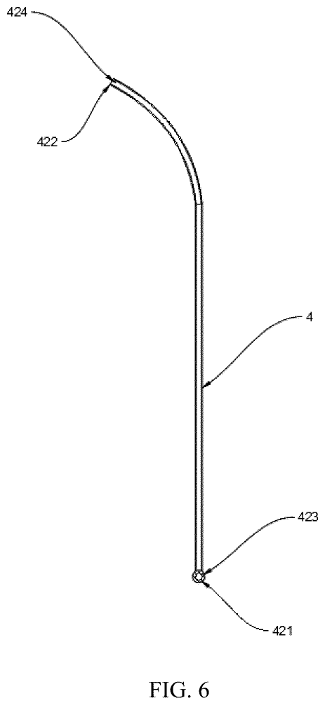

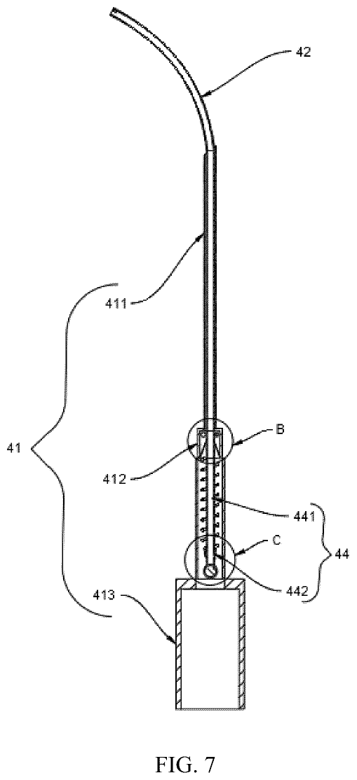

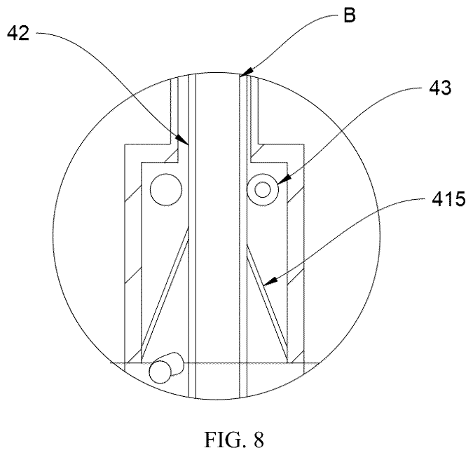

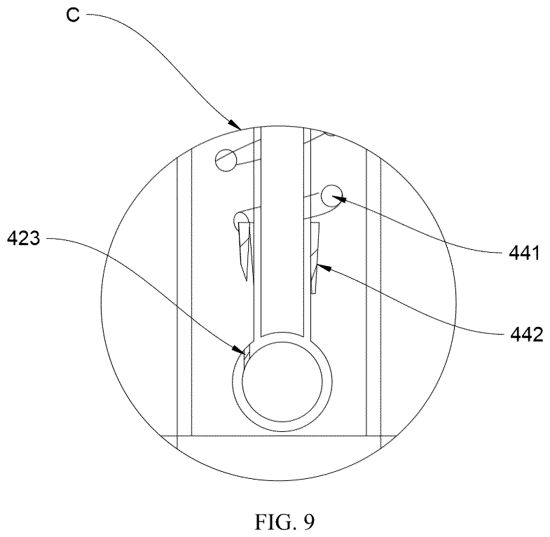

Referring to FIGS. 1-9, an embodiment of the present disclosure provides a drug infusion device for tumor interventional therapy, including a fixing base 1, a rotating support frame 2, a locking assembly 3, an infusion apparatus 4 and a storage table.

The fixing base 1 is circumferentially fixed to an outer side of the human body. The fixing base 1 includes a flexible bottom plate 11, a sliding rod 12, a buckle strap 13 and two flexible straps 14. The two flexible straps 14 are fixedly connected to two ends of the flexible bottom plate 11, and the sliding rod 12 is fixedly connected to the flexible bottom plate 11. The buckle strap 13 is fixedly connected to one of the flexible straps 14 for locking the two flexible straps 14. When performing drug infusion on a patient, the fixing base 1 needs to be fixed adjacent to a patient's tumor site. Due to the complexity and nearly irregular shape of the human body, the flexible straps 14 are employed here as a fixation method, causing the sliding rod 12 in proximity to the patient's tumor site for fixation. For instance, when a patient's tumor is located in the head, the fixing base 1 can be fixed to the patient's forehead. If the tumor is near the patient's neck, the fixing base 1 can be fixed to the patient's chest. The flexible straps 14 can be fastened using a buckle locking mechanism or fixed with hook and loop fastener. In this embodiment, the flexible bottom plate 11 is constructed from leather material and designed in a strip-type plate to conform snugly to the patient's body surface. The flexible straps 14 are made of spandex material, offering moderate fastening strength to prevent patient discomfort. Naturally, in alternative embodiments, both the flexible bottom plate 11 and flexible strap 14 could be fabricated using materials such as rubber, silk, or elastic cloth, and may feature alternative shapes including square or oval configurations. The sliding rod 12 can be constructed using elastic metallic materials or plastic materials, ensuring both the stability of the fixing base 1 and excellent adaptability to the irregular contours of the human body.

The rotating support frame 2 is slidably connected to the fixing base 1. The rotating support frame 2 includes a top support frame 21, a rotating table 22 and a clamping assembly 23, one end of the top support frame 21 is slidably connected to the fixing base 1, and the other end is rotatably connected to the rotating table 22. The clamping assembly 23 is fixedly connected to the rotating table 22. The top support frame 21 is specifically mounted on the sliding rod 12, allowing it to move on the sliding rod 12 towards two ends of the sliding rod 12, or rotate around the sliding rod 12, thereby facilitating the position adjustment of one end of the top support frame 21 to be further close to the tumor site.

Specifically, the top support frame 21 includes a four-link structure 211, a sliding sleeve I 212, and a sliding sleeve II 213. The sliding sleeve I 212 is movably connected to the sliding rod 12 of the fixing base 1, allowing it to freely move on the sliding rod 12 or rotate around the sliding rod 12. The sliding sleeve II 213 is rotatably connected to the sliding rod 12 of the fixing base 1. Two ends of the four-link structure 211 are rotatably connected to the sliding sleeve I 212 and the sliding sleeve II 213. A movable rod of the four-link structure 211 is rotatably connected to the rotating table 22. In this embodiment, the four-link structure 211 is formed by a drive rod, a movable rod, a driven rod and the sliding rod 12 together, in which one end of the drive rod is rotatably connected to the sliding sleeve I 212, the other end is rotatably connected to one end of the movable rod, the other end of the movable rod is rotatably connected to one end of the driven rod, and the other end of the movable rod is rotatably connected to the sliding sleeve II 213. In addition, a locking bolt 321 is mounted on the drive rod and/or the driven rod. After adjusting the position of the movable rod, the four-link structure 211 is fixed to the sliding sleeve I 212 and the sliding sleeve II 213 using the locking bolt 321. The top support frame 21 can be rotated on the sliding rod 12 through the sliding sleeve I 212 and the sliding sleeve II 213, for the adjustment of a distance between the movable rod and the patient's body surface.

The clamping assembly 23 includes a sliding support frame 231, a drive arm 232 and clamp plates 233, and the sliding support frame 231 is fixedly connected to the rotating table 22. The clamp plates 233 are slidably connected to the sliding support frame 231. The drive arm 232 is mounted on the sliding support frame 231 for driving the clamp plates 233 to clamp or release, realizing the clamping of the infusion apparatus 4, and facilitating the replacement of the infusion apparatuses 4 with different sizes. Specifically, the clamp plates 233 can be formed by two independent clamp plates 233 arranged opposite each other and rotatably connected to the sliding support frame 231, or V-shaped clamp plates 233 rotatably connected to the sliding support frame 231. The drive arm 232 may select an electrically adjustable or a manually adjustable drive arm 232. For instance, an electric telescopic rod can be employed, with two ends of the electric telescopic rod located on the two clamp plates 233, driving the two clamp plates 233 to rotate relative to each other, thereby achieving clamping or releasing operations.

The locking assembly 3 is used for locking the rotating support frame 2 with the fixing base 1. The locking assembly 3 includes a direction locking structure 31 and position locking structures 32. The direction locking structure 31 can lock the top support frame 21 after the top support frame 21 is adjusted to a suitable direction, preventing the top support frame 21 from rotating relative to the sliding rod 12. The direction locking structure 31 includes an outer spline 311 and a locking block 312, the outer spline 311 is slidably connected to the fixing base 1, and an inner spline groove bonded with the outer spline 311 is disposed inside the sliding sleeve I 212. Two sides of the locking block 312 are slidably connected to the outer spline 311 and the fixing base 1. In actual operation, an angle of the top support frame 21 is manually adjusted, causing the rotating table 22 to be located exactly on the outer side of the human body to be punctured, thereby bonding the outer spline 311 into the sliding sleeve I 212.

The position locking structure 32 can lock the top support frame 21 after the top support frame 21 is adjusted to an appropriate position, preventing the top support frame 21 from sliding on the sliding rod 12. For instance, when the rotating table 22 is located precisely on the outer side of the body surface corresponding to the patient's tumor site, the top support frame 21 is fully locked by the direction locking structure 31 and the position locking structure 32. The position locking structure 32 includes the locking bolt 321 and a limiting block 322. The locking bolt 321 passes through the sliding sleeve I 212 and is screwed with the sliding sleeve I 212, and the limiting block 322 is fixedly connected to the locking bolt 321. By rotating the locking bolt 321, the limiting block 322 is abutted against the sliding rod 12 to lock the sliding sleeve I 212, preventing the top support frame 21 from moving along the sliding rod 12, thereby achieving the locking of the top support frame 21. In practical applications, in order to ensure uniform force distribution on two sides of the sliding sleeve I 212, the position locking structures 32 can be arranged on two sides of the sliding sleeve I 212.

The infusion apparatus 4 is mounted on the rotating support frame 2 for infusing drug. The infusion apparatus 4 includes a puncture needle 41, a flow guide needle 42, a driver 43, a limiting stopper 44 and a sealing structure 45. The puncture needle 41 is used for puncturing a patient's blood vessel. The flow guide needle 42 is arranged inside the puncture needle 41. The driver 43 is used for driving the flow guide needle 42 into or out of the blood vessel. The limiting stopper 44 is used for limiting the flow guide needle 42, and causing at least one end of the flow guide needle 42 to be located in the puncture needle 41. The sealing structure 45 is arranged inside a gap between the flow guide needle 42 and the puncture needle 41. During the tumor interventional therapy drug infusion to the patient, a puncture location is determined based on the patient's tumor site. The blood vessel is punctured using the puncture needle 41, and the flow guide needle 42 is extended along the blood vessel to the tumor site, achieving precise drug infusion to the patient's tumor site. The puncture needle 41 can be clamped by the clamping assembly 23. Under the combined action of the rotating table 22 and the sliding support frame 231, the puncture needle 41 can move relative to the rotating support frame 2, and the rotating support frame 2 can provide good protection for the puncture needle 41, preventing displacement of the puncture needle 41 due to external forces, and avoiding injury to the patient.

The flow guide needle 42 includes a limiting end 421 and an outlet end 422. A through hole is disposed at the limiting end 421. A valve 423 is arranged at one end of the through hole. A fiber optic sensor 424 is mounted at the outlet end 422. During the puncture procedure, the flow guide needle 42 and the puncture needle 41 are not communicated due to the closure of the valve 423 within the through hole, preventing blood reflux and minimizing injury to the patient. During drug infusion, the valve 423 is opened by the limiting stopper 44 to establish a communication with the puncture needle 41, thereby allowing direct drug infusion to the patient's tumor site. The fiber optic sensor 424 can include a pressure sensor to measure blood pressure and determine whether the patient's tumor site has been reached. In other embodiments, a miniature probe can also be mounted at the outlet end 422 to more accurately determine the tumor site.

The limiting stopper 44 includes a spring 441 and a jacking block 442. The spring 441 is mounted inside the puncture needle 41, and an inner diameter of the spring 441 is smaller than a maximum outer diameter of the limiting end 421. The jacking block 442 is mounted on an inner side of the spring 441 for opening the valve 423. The flow guide needle 42 passes through an interior of the spring 441 and enters the human blood vessel through the puncture needle 41, allowing direct access to the patient's tumor site, thereby realizing more precise drug infusion, and enhancing the therapeutic efficacy of the drug. By arranging the spring 441 and the jacking block 442, a length of the flow guide needle 42 entering the blood vessel can be adjusted, allowing it to accommodate tumor sites at varying depths.

The puncture needle 41 includes a needle head 411, a fixing block 412, a connecting pipe 413, a handheld portion 414 and guide pipes 415. The needle head 411 is fixedly connected to and communicated with the fixing block 412, and the needle head 411 can be directly pierced into the human blood vessel, establishing a direct communication with the blood vessel. The handheld portion 414 is fixedly connected to the fixing block 412, and the handheld portion 414 is typically arranged as a butterfly-shaped piece and can be made of plastic or metal materials. The connecting pipe 413 is communicated with the fixing block 412. An inner diameter of the connecting pipe 413 is typically larger than an outer diameter of the needle head 411, allowing a communication with a catheter. In this embodiment, the catheter can be arranged in two parts, including a front-end catheter and a rear-end catheter. One end of the front-end catheter is communicated to the connecting pipe 413, a one-way guide port is arranged at the other end, and the one-way guide port is fixedly connected to the rotating support frame 2. One end of the rear-end catheter is communicated to the one-way guide port, and the other end is connected to the storage table. The guide pipes 415 are fixedly connected inside the connecting pipe 413, and the flow guide needle 42 passes through the guide pipes 415 to achieve precise positioning.

The driver 43 includes a knob, a connecting rod, a sealing ring and two roller wheels, and the knob is mounted on an outer wall of the connecting pipe 413. One end of the connecting rod is fixedly connected to the knob, the other end passes through the connecting pipe 413 and is fixedly connected to one of the roller wheels, the other roller wheel is rotatably connected inside the connecting pipe 413, and the two roller wheels are arranged in parallel and symmetrically fitted on two sides of the flow guide needle 42. By rotating the knob, the connecting rod and the roller wheels are driven to rotate synchronously. Under the combined action of the two roller wheels, the flow guide needle 42 is driven to enter or exit the human blood vessel. A sealing ring is arranged on an outer side of the connecting rod, avoiding drug leakage during the driving process.

The storage table is communicated with the infusion apparatus 4 using the catheter for storing drug. The storage table can be a medical operating table or a simple object stage, with a container inside for storing drugs. The container can be connected to the puncture needle 41 through a medical pump to infuse drugs into the human body, or drugs can be delivered into the human body by manual compression.

An operating principle is as follows. This embodiment provides the drug infusion device for tumor interventional therapy. According to the blood vessel to be punctured in the patient, the corresponding specification of the infusion apparatus 4 is selected, and the infusion apparatus 4 is clamped using the clamping assembly 23. The fixing base 1 is worn on the outer side of the human body by being fixed to each other through two flexible straps 14, and the position of the rotating support frame 2 is manually adjusted, causing the infusion apparatus 4 clamped by the clamping assembly 23 to close to the site to be punctured. At this time, the infusion apparatus 4 is evacuated to ensure that an interior of the infusion apparatus 4 is filled with tumor therapeutic drugs, and the infusion apparatus 4 is pierced into the human blood vessel through the puncture needle 41. At this time, since the flow guide needle 42 is not communicated to the puncture needle 41, blood will not flow back through the puncture needle 41. To further ensure the stability of the puncture needle 41, medical adhesive tape, gauze, and the like can be used for additional fixation of the puncture needle 41. The top support frame 21 is fixed to the sliding rod 12 through the locking assembly, avoiding the movement of the top support frame 21. At this time, since the rotating table 22 can rotate relative to the top support frame 21, and the clamp plates 233 can move relative to the sliding support frame 231, the puncture needle 41 can move freely within a local range without interfering with the top support frame 21, avoiding damage to the puncture site. Moreover, the top support frame 21 can provide a good protective effect for the puncture needle 41. Even with external forces, it can avoid the impact on the puncture and effectively protect the patient's safety. During drug infusion, the driver 43 moves the flow guide needle 42 into the blood vessel, causing the outlet end 422 of the flow guide needle42 to close to the tumor site. At this time, the limiting end 421 of the flow guide needle 42 is abutted against the spring 441, and the jacking block 442 is driven to open the valve 423, causing the flow guide needle 42 to be communicated with the puncture needle 41, thereby ensuring drug infusion through the flow guide needle 42 to the tumor site. In this embodiment, accurate drug infusion for tumor interventional therapy is not only achieved, but also adjustable components including the flow guide needle 42 and top support frame 21 are arranged, causing the infusion arrangement to be applicable to the majority of the population, while effectively preventing drug waste during the infusion, thereby reducing therapeutic costs.

Through the description of the above implementation methods, a person skilled in the art can clearly understand that each implementation method can be realized with the help of software and necessary general hardware platforms, and of course, it can also be realized through hardware. Based on this understanding, the part of the above technical solution essentially or contributes to the related art can be embodied in the form of a software product. The computer software product can be stored in a computer-readable storage medium, such as a read-only memory (ROM)/a random access memory (RAM), magnetic disks, optical discs, etc., including several instructions to cause a computer device, which can be a personal computer, server, or network device, to execute the methods of various embodiments or certain parts of the embodiments.

Finally, it is to be noted that the above embodiments are only used for stating the technical solutions of the present disclosure, but not limitation. Although the present disclosure is described in detail by reference to the foregoing embodiments, for those ordinary skill in the art, it is to be understood that the technical solutions described in the above embodiments may still be modified, or some or all of the technical features may be equivalently replaced. However, these modifications or substitutions do not deviate the essence of the corresponding technical solutions from the scope of the technical solutions of each embodiment of the present disclosure.

1. A drug infusion device for tumor interventional therapy, comprising:
a fixing base (1), configured to be circumferentially fixed to an outer side of the human body adjacent to a tumor site;
a rotating support frame (2), slidably connected to the fixing base (1);
a locking assembly (3), used for locking the rotating support frame (2) with the fixing base (1); and
an infusion apparatus (4), mounted on the rotating support frame (2) for infusing drug; and
wherein the fixing base (1) comprises a flexible bottom plate (11), an elastic sliding rod (12), a buckle strap (13) and two flexible straps (14); the two flexible straps (14) are fixedly connected to two ends of the flexible bottom plate (11), and the elastic sliding rod (12) is fixedly connected to the flexible bottom plate (11); and the buckle strap (13) is fixedly connected to one of the flexible straps (14) for locking the two flexible straps (14);
wherein the rotating support frame (2) comprises a top support frame (21), a rotating table (22) and a clamping assembly (23), one end of the top support frame (21) is slidably connected to the fixing base (1), and the other end is rotatably connected to the rotating table (22); and the clamping assembly (23) is fixedly connected to the rotating table (22); and
wherein the top support frame (21) comprises a four-link structure (211), a sliding sleeve I (212), and a sliding sleeve II (213); the sliding sleeve I (212) is movably connected to the fixing base (1); the sliding sleeve II (213) is rotatably connected to the fixing base (1); two ends of the four-link structure (211) are rotatably connected to the sliding sleeve I (212) and the sliding sleeve II (213); the four-link structure (211) is formed by a drive rod (2111), a movable rod (2112), a driven rod (2113) and the elastic sliding rod (12) together, in which one end of the drive rod (2111) is rotatably connected to the sliding sleeve I (212), the other end of the drive rod (2111) is rotatably connected to one end of the movable rod (2112), the other end of the movable rod (2112) is rotatably connected to one end of the driven rod (2113), and the other end of the driven rod (2113) is rotatably connected to the sliding sleeve II (213); the movable rod (2112) of the four-link structure (211) is rotatably connected to the rotating table (22); and a length of the drive rod (2111) is different from a length of the driven rod (2113);
wherein the infusion apparatus (4) comprises a puncture needle (41), a flow guide needle (42), a driver (43), a limiting stopper (44) and a sealing structure (45); the puncture needle (41) is used for puncturing a patient's blood vessel; the flow guide needle

(42) is arranged inside the puncture needle (41); the driver (43) is used for driving the flow guide needle (42) into or out of the blood vessel; the limiting stopper (44) is used for limiting the flow guide needle (42), and causing at least one end of the flow guide needle (42) to be located in the puncture needle (41); and the sealing structure (45) is arranged inside a gap between the flow guide needle (42) and the puncture needle (41);
wherein the flow guide needle (42) comprises a limiting end (421) and an outlet end (422); a through hole is disposed at the limiting end (421); a valve (423) is arranged at one end of the through hole; and a fiber optic sensor (424) is mounted at the outlet end (422);
wherein the limiting stopper (44) comprises a spring (441) and a jacking block (442); the spring (441) is mounted inside the puncture needle (41), and an inner diameter of the spring (441) is smaller than a maximum outer diameter of the limiting end (421);
and the jacking block (442) is mounted on an inner side of the spring (441) for opening the valve (423).

2.-4. (canceled)

5. The drug infusion device for tumor interventional therapy according to claim 1, wherein the clamping assembly (23) comprises a sliding support frame (231), a drive arm (232) and clamp plates (233), and the sliding support frame (231) is fixedly connected to the rotating table (22); the clamp plates (233) are slidably connected to the sliding support frame (231); and the drive arm (232) is mounted on the sliding support frame (231) for driving the clamp plates (233) to clamp or release.

6. The drug infusion device for tumor interventional therapy according to claim 1, wherein the locking assembly (3) comprises a direction locking structure (31) and position locking structures (32).

7.-9. (canceled)

10. The drug infusion device for tumor interventional therapy according to claim 1, wherein the puncture needle (41) comprises a needle head (411), a fixing block (412), a connecting pipe (413), a handheld portion (414) and guide pipes (415); the needle head (411) is fixedly connected to and communicated with the fixing block (412); the handheld portion (414) is fixedly connected to the fixing block (412); the connecting pipe (413) is communicated with the fixing block (412); the guide pipes (415) are fixedly connected inside the connecting pipe (413).
